(19) United States
(12) Patent Application Publication
(10) Pub. No.: US 2026/0272987 A1
(43) Pub. Date: Sep. 17, 2026

(54) METHOD OF TREATING OLFACTORY DISORDER

(71) Applicant: Taichung Veterans General Hospital, Taichung (TW)

(72) Inventor: RONG-SAN JIANG, Taichung (TW)

(73) Assignee: Taichung Veterans General Hospital, Taichung (TW)

(21) Appl. No.: 19/075,790

(22) Filed: Mar. 11, 2025

(51) Int. Cl. A61K 35/19; A61K 31/728; A61K 35/16; A61P 27/00

(52) U.S. Cl. CPC A61K 35/19; A61K 31/728; A61K 35/16; A61P 27/00

(57) ABSTRACT

A method of treating an olfactory disorder can be performed on a patient after the patient receives only local facial anesthesia, and the method essentially includes injecting a plasma composition into the patient's nasal cavity so that the patient's olfactory functions can be effectively improved or enhanced after the injection, thereby achieving the effect of treating the patient's olfactory disorder.

### TECHNICAL FIELD

The present invention relates to a method of disease treatment. More particularly, the invention relates to a method of treating an olfactory disorder.

### BACKGROUND

Olfactory dysfunction, also known as olfactory disorder, refers to a change, degradation, or total loss of one's smell detection and recognition ability. The causes of olfactory dysfunction abound, including, for example, allergic rhinitis, a nasal tumor, a structural anomaly in the nasal cavity, viral infection, and bacterial infection. Patients who are confirmed cases of novel coronavirus pneumonia tend to have symptoms of anosmia, i.e., a loss of the sense of smell, and according to worldwide statistics, about 20-85% of patients infected with a novel coronavirus suffer from anosmia. While some patients experience anosmia only temporarily, studies have shown that nearly 46% of those infected with a novel coronavirus are experiencing a degradation or loss of olfactory functions or parosmia even 12 to 18 months after the infection, with about 7% of the patients losing their olfactory ability completely.

Currently, due to a lack of special treatment methods for olfactory dysfunction, it is common practice to deal with olfactory dysfunction by providing improvement methods targeted at specific causes of it. For example, if a patient's olfactory dysfunction is caused by bacterial infection, an antibiotic will be administered to the patient in an attempt to restore the patient's sense of smell by resolving the bacterial infection. Alternatively, it may be feasible to administer a steroid to the patient or provide the patient with olfactory training when symptoms of olfactory dysfunction appear. In either case, however, the prognosis is not as good as expected.

### SUMMARY

The primary objective of the present invention is to provide a method for treating an olfactory disorder, wherein the method can treat a patient's olfactory disorder by injecting a plasma composition into the patient's nasal cavity without general anesthesia.

To achieve the foregoing objective, the present invention discloses a method of treating an olfactory disorder. The method includes administering, by nasal injection, an effective amount of a plasma composition into the nasal cavity of a patient with a symptom of olfactory dysfunction, in order for the plasma composition, or more particularly its active ingredients, to act on the patient's nasal cavity and thereby improve the patient's olfactory functions. The plasma composition contains a hyaluronic acid and plasma rich in platelets.

The hyaluronic acid and the platelet-rich plasma constitute the active ingredients of the plasma composition, and the hyaluronic acid and the platelet-rich plasma are in a volume ratio of 1:1-1.5.

A single dose of injection of the plasma composition is at least 5 mL.

In one embodiment of the present invention, the platelet-rich plasma is obtained from the blood of the patient on which the method of the invention is to be performed.

In another embodiment of the present invention, the nasal injection is carried out after local anesthesia is performed on a portion of the patient's nasal cavity and/or on the intended injection area in the nasal cavity. For example, the local anesthesia includes injecting an anesthetic directly into a portion of the nasal cavity and/or bringing an anesthetic into contact with a predetermined area in the nasal cavity.

In yet another embodiment of the present invention, the nasal injection is carried out after the olfactory sulci in the patient's nasal cavity are opened with a tool, so that the plasma composition can be injected precisely into predetermined portions in the nasal cavity.

The predetermined portions in the nasal cavity that are to receive the nasal injection are the mucous membrane on the portions of the nasal septum that are opposite the middle upper halves of the middle nasal conchae in the nasal cavity and the inner sides of the middle nasal conchae.

### BRIEF DESCRIPTION OF THE DRAWINGS

FIG. 1 is a photo showing how a cotton pad impregnated with an anesthetic is put into a patient's nasal cavity.

FIG. 2 is a photo showing the positions where an anesthetic is injected.

FIG. 3 is photos showing how sub-step 301 in an embodiment of the present invention is performed.

FIG. 4 is a photo showing how sub-step 302 in an embodiment of the present invention is performed.

FIG. 5 is photos showing the positions where a plasma composition is injected.

### DETAILED DESCRIPTION OF THE EMBODIMENTS

The present invention discloses a method of treating an olfactory disorder. The method can be performed on a patient after the patient receives only local facial anesthesia, and the method essentially includes injecting a plasma composition into the patient's nasal cavity so that the patient's olfactory functions are improved about 60 days after the nasal injection. The plasma composition contains a hyaluronic acid and platelet-rich plasma.

In one embodiment of the present invention, the patient has a symptom of anosmia. For example, the patient is a confirmed case of novel coronavirus pneumonia, or the patient's olfactory disorder is caused by a traumatic injury to the head and/or the neck.

In one embodiment of the present invention, the platelet-rich plasma is isolated from the patient's own blood.

In one embodiment of the present invention, the operator of the nasal injection is a physician or a person meeting the requirements of applicable regulations of the place where the nasal injection is performed. The nasal injection includes injecting the plasma composition into predetermined portions in the patient's nasal cavity with a syringe or an injector.

The predetermined portions in the nasal cavity include portions opposite the middle upper halves of the middle nasal conchae.

In yet another embodiment of the present invention, the nasal injection is conducted after local anesthesia is performed on a portion of the patient's nasal cavity or on the intended injection area in the nasal cavity.

The term “platelet-rich plasma (PRP)” is defined herein as a plasma fraction that has a platelet concentration higher than a baseline, and that is isolated from the blood of the patient on which the method of the present invention is to be performed. The platelet-rich plasma contains a variety of growth factors in addition to a high concentration of platelets. Generally, the platelet-rich plasma is a plasma fraction isolated by centrifuging the patient's blood and then removing the red blood cells (erythrocytes) and white blood cells (leukocytes) in the centrifuged blood.

The term “hyaluronic acid (HA),” also referred to as hyaluronan, refers to glycosaminoglycan that has a high molecular weight and that can function as the backbone of the proteoglycans in the extracellular matrix.

The term “olfactory dysfunction,” also referred to as olfactory disorder, refers to a loss or degradation of the sense of smell. Olfactory dysfunction has many potential causes, and the olfactory dysfunction referred to herein includes that caused by a virus, a traumatic injury, or other clinically known causes of disease.

The term “local anesthesia” refers to a procedure by which only a predetermined portion of a patient's body is anesthetized, i.e., rendered insensible to pain or completely insensible, while the patient remains conscious. Generally, local anesthesia is carried out by, for example, injection of an anesthetic and/or bringing an anesthetic into contact with a predetermined area of the patient's body.

To shed more light on the technical features of the present invention and their effects, some embodiments of the invention and experiments are described in detail below with reference to the accompanying drawings.

One embodiment of the present invention discloses a method of treating an olfactory disorder induced by novel coronavirus pneumonia. The method is used to treat a patient with olfactory dysfunction and includes the following steps:
Step 1: A plasma composition is prepared.
Step 1 includes the following sub-steps:

Sub-step 101: Blood is collected from the patient. Generally, at least 6 cc of blood is required.

Sub-step 102: The collected blood is added with an anticoagulant and then centrifuged in order to isolate platelet-rich plasma. The centrifugation may be conducted by any centrifugation method known to a person of ordinary skill in the art, such as by putting the blood into a centrifuge tube and performing the intended centrifugation process with a centrifuge. The anticoagulant may be sodium citrate or any other medically acceptable preparation for reducing or preventing the coagulation of blood, and the volume ratio of the anticoagulant to the blood is about 10:1.

Sub-step 103: The platelet-rich plasma and a hyaluronic acid are thoroughly mixed in a predetermined volume ratio to obtain the plasma composition, whose volume is about 5 mL and which contains about 40 mg of the hyaluronic acid. The predetermined volume ratio of the platelet-rich plasma to the hyaluronic acid is 1-1.5: 1. For example, 3 mL of the platelet-rich plasma is mixed with 2 mL of the hyaluronic acid. The term “thoroughly mixed” refers to the platelet-rich plasma and the hyaluronic acid being mixed by repeatedly agitating the container holding the platelet-rich plasma and the hyaluronic acid.

Step 2: Local anesthesia is performed.

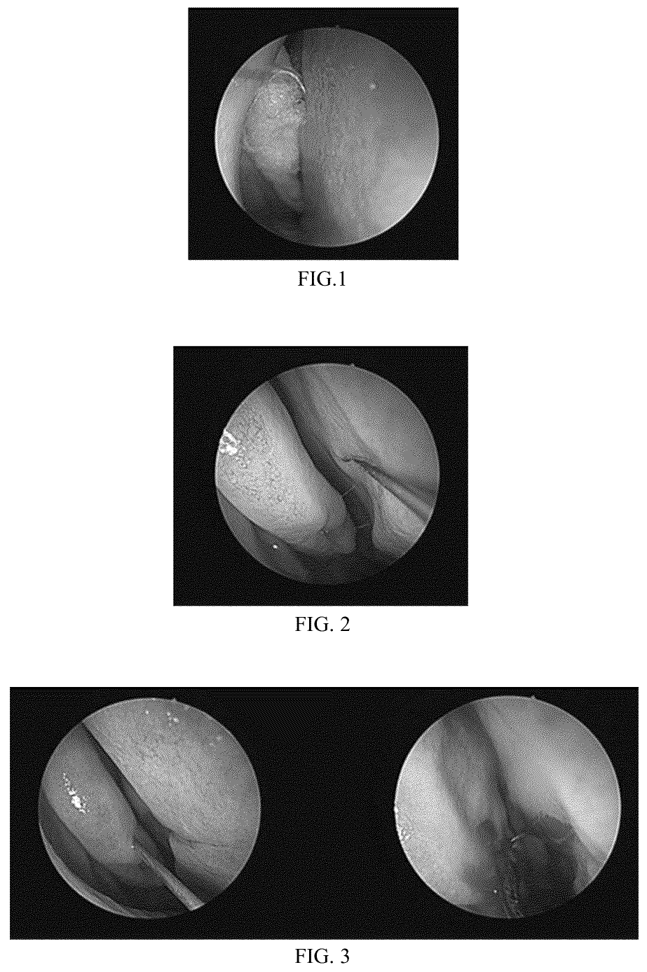

Step 2 involves performing local anesthesia on a portion of the patient's nasal cavity and on the intended injection area in the nasal cavity by a method of local anesthesia that is well known in the art. More specifically, a cotton pad impregnated with an anesthetic is put into the patient's nasal cavity (see FIG. 1) and is removed after a predetermined time, and then an anesthetic is injected into the patient's nasal septum and middle nasal conchae (see FIG. 2).

Step 3: Nasal injection is performed.

Step 3 includes the following sub-steps:

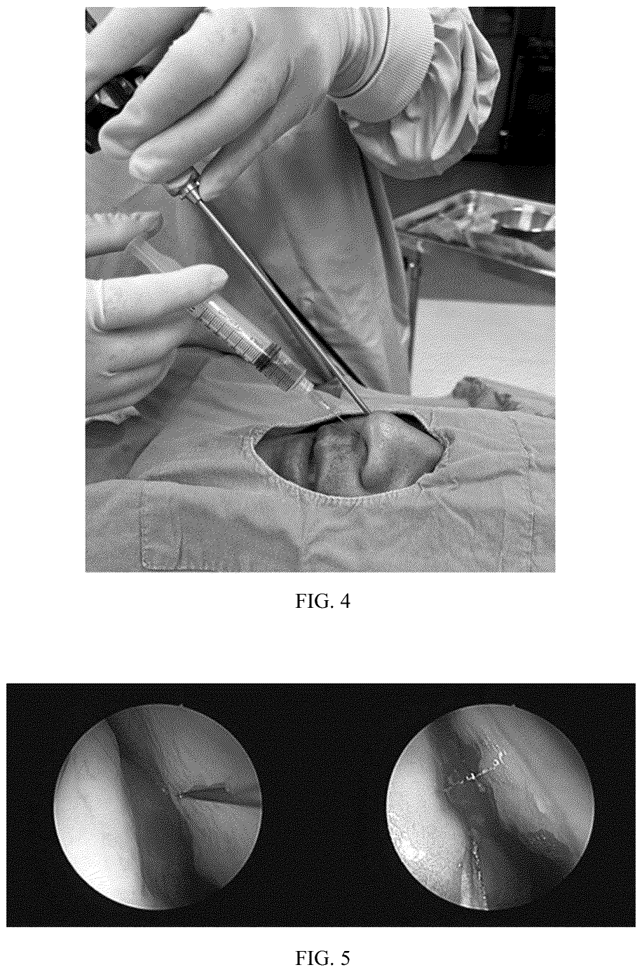

Sub-step 301: The olfactory sulci in the patient's nasal cavity are opened with a tool, as shown in FIG. 3.

Sub-step 302: The plasma composition prepared in step 1 is put into an injector and then injected through the injector into the mucous membrane on the portions of the nasal septum that are opposite the middle upper halves of the middle nasal conchae in the patient's nasal cavity and into the inner sides of the middle nasal conchae (see FIG. 4 and FIG. 5).

Another embodiment of the present invention discloses another version of the aforesaid method of treating an olfactory disorder induced by novel coronavirus pneumonia. The steps of this version are generally identical to those in the previous embodiment except that the treating procedure of step 1 is carried out with a commercially available blood isolation kit. More specifically, about 6 mL of the blood collected from the patient is put into the blood collection tube in the commercially available blood isolation kit, and then the blood collection tube is placed in a centrifuge and subjected to centrifugation at 1500 g for 5 minutes. After the centrifugation, the blood in the blood collection tube has been stratified, with the upper layer being a blood cell layer, and the lower layer being a platelet-rich plasma layer. The hyaluronic acid is subsequently added into the blood collection tube such that the hyaluronic acid lies on top of the platelet-rich plasma layer, and then the blood collection tube is repeatedly turned upside side and/or horizontally rolled until the platelet-rich plasma and the hyaluronic acid are determined, by visual inspection, to be in a homogeneous state.

To verify the effect of the method of the present invention, a plurality of patients who were suffering from olfactory dysfunction caused by a traumatic injury to the head were recruited and divided into two groups each given a different treatment. More specifically, one of the groups was treated by the method of the invention for treating a novel coronavirus pneumonia-induced olfactory disorder, and the other group was treated by a conventional method, i.e., by administering a drug such as a steroid or zinc tablets to the patients and providing the patients with olfactory training. Three months after the treatments, the patients in each group were evaluated in terms of improvement of their olfactory functions. Not more than 30% of the patients receiving the conventional treatment showed signs of improvement in olfactory function. By contrast, about 85% of the patients treated by the method of the invention for treating a novel coronavirus pneumonia-induced olfactory disorder felt improvement, and it can be known from olfactory function test results that patients with an olfactory threshold of 50% or above thereof experienced a significant improvement in olfactory function.

The foregoing results prove that the method of the present invention for treating an olfactory disorder caused by a traumatic injury to the head is indeed capable of effectively treating or improving symptoms of olfactory dysfunction and thereby enhancing the patients'life quality to a great extent. The method of the invention for treating a novel coronavirus pneumonia-induced olfactory disorder does not require general anesthesia and is a highly safe treatment method as far as patients are concerned.

1. A method of treating an olfactory disorder, comprising: administering an effective amount of a plasma composition to a patient's nasal cavity by nasal injection, wherein:
the plasma composition contains a hyaluronic acid and platelet-rich plasma; and
the patient has a symptom of olfactory dysfunction.

2. The method of treating the olfactory disorder according to claim 1, wherein the platelet-rich plasma is isolated from the patient's blood.

3. The method of treating the olfactory disorder according to claim 1, comprising:
step A: performing local anesthesia on a portion of the patient's nasal cavity;
step B: opening olfactory sulci in the patient's nasal cavity with a tool; and
step C: injecting the plasma composition into middle-to-upper portions of the patient's middle nasal conchae.

4. The method of treating the olfactory disorder according to claim 3, wherein the step A comprises:
sub-step A1: putting cotton impregnated with an anesthetic into the patient's nasal cavity; and
sub-step A2: injecting the anesthetic into the patient's nasal septum and middle nasal conchae.

5. The method of treating the olfactory disorder according to claim 3, wherein in the step C, the plasma composition is injected into a mucous membrane on portions of the patient's nasal septum that are opposite middle upper halves of the middle nasal conchae and into inner sides of the middle nasal conchae.

6. The method of treating the olfactory disorder according to claim 1, wherein the hyaluronic acid and the platelet-rich plasma constitute main active ingredients of the plasma composition.

7. The method of treating the olfactory disorder according to claim 6, wherein the hyaluronic acid and the platelet-rich plasma are in a volume ratio of 1:1-1.5.

8. The method of treating the olfactory disorder according to claim 1, wherein the plasma composition is injected in a dose of at least 5 ml.
